(19) United States
(12) Patent Application Publication
(10) Pub. No.: US 2011/0307038 A1
(43) Pub. Date: Dec. 15, 2011

(54) BARRIERS FOR CONTROLLED USE OF THERAPY SYSTEMS

(75) Inventors: DAVID R. STIEHR, Pleasanton, CA (US); Allison Mahan Sojka, Alameda, CA (US); Mark H. Lowe, Danville, CA (US)

(73) Assignee: CoolSystems, Inc., Alameda, CA (US)

(21) Appl. No.: 13/157,150

(22) Filed: Jun. 9, 2011

Related U.S. Application Data
(60) Provisional application No. 61/354,020, filed on Jun. 11, 2010.

(51) Int. Cl. A61F 7/00

(52) U.S. Cl. 607/104

(57) ABSTRACT

A therapy system barrier. In various embodiments, the barrier is a liner sized to cover a surface of a housing having a control interface and at least one other face of the housing. In various embodiments, the therapy system barrier hinders proper operation of a related therapy system until the barrier is removed or modified. The barrier may interact with one or more therapy system components to hinder use of the involved therapy system component until a user performs a desired operation. When the barrier is in place, electrical and/or mechanical components of the device may be rendered at least partially inoperative. Also disclosed are methods of using the barrier.

### CROSS REFERENCE TO RELATED APPLICATIONS

This application claims priority to U.S. Provisional Application No. 61/354,020 filed Jun. 11, 2010, entitled, “BARRIERS FOR CONTROLLED USE OF THERAPY SYSTEMS.”

### INCORPORATION BY REFERENCE

All publications and patent applications mentioned in this specification are herein incorporated by reference to the same extent as if each individual publication or patent application was specifically and individually indicated to be incorporated by reference.

### FIELD OF THE INVENTION

This application relates to barriers used to inhibit, restrict or control the use of therapy systems. The barriers may also provide information about how to use a particular therapy system or limit the ability of a user to control or access the therapy system.

### BACKGROUND OF THE INVENTION

Temperature therapy systems exist in a number of different forms. In general, there is a control unit, a connector hose, a wrap and a power source (i.e., battery or external electric power). The wrap is applied to the portion of the mammal's body to receive therapy. The control unit is used to modulate a fluid circulated through the wrap to achieve the desired therapeutic result.

While the use of individual therapy systems may require specific steps, the general steps for setting up and operating a therapy system include: attaching the wrap to the mammal, connecting the wrap to the control unit or circulation system (if separate from the control unit), preparing the reservoir, connecting the control unit to an appropriate source of power and then operating the control unit to provide the desired therapy. However, improper set up or operation of the system can diminish the effectiveness of the therapy being provided, or impair the functionality of one or more components of the system. In some cases, improper set up can result in damage to system components or even user injury. One example of improper set up is operation of the system without any fluid in the reservoir. Another example would be improper connection of system components. Still another example is not properly seating a connector between the wrap and the connector hose or to the control unit. Still another example is user error in system operation that results in a therapy system use that does not comply with, for example, the intended use either by duration of therapy or temperature of therapy.

What is needed are improved devices and techniques for, among other things, ensuring the proper set up and operation of therapy systems. While the examples above and that follow relate to a cold therapy system, the inventive methods and devices disclosed herein may be used for other therapy systems, such as, heating therapy systems or pneumatic systems that provide pressure to the body portion, alone or in combination with a temperature based therapy system.

### BRIEF SUMMARY OF THE INVENTION

In summary, one aspect of the present invention is directed to a medical device including a housing, an access to an interior chamber of the housing, a control interface on a surface of the housing, and a liner. In various embodiments, the liner is sized to cover at least a portion of the surface of the housing and at least one other face of the housing. In various embodiments, when the liner is positioned to cover at least a portion of the surface of the housing, the access is rendered inoperative. In various embodiments, when the liner is positioned to cover at least a portion of the surface of the housing, the access is rendered at least partially inoperative. In various embodiments, when the liner is positioned to cover at least a portion of the surface of the housing, the access is rendered completely inoperative. In various embodiments, the liner is configured to render the respective component and/or control unit completely inoperative. In various embodiments, the liner is configured to hinder operation of the access when positioned to cover the surface of the housing.

Various aspects of the invention are directed to a control unit for a patient therapy system including a housing containing the control unit, a chamber within the housing, an interface on the housing used to interact with the control unit, a moveable panel on the housing in communication with the chamber, and a liner. In various embodiments, the liner is sized to conform to and at least partially cover a surface of the housing. In various embodiments, the liner is sized to conform to and at least partially cover at least two adjacent surfaces of the housing. In various embodiments, when the liner is positioned on the housing surface, the control interface, the moveable panel, or both is rendered at least partially inoperative. In various embodiments, the control interface, panel, or both is rendered completely inoperative. In various embodiments, the liner is configured to hinder operation of a component, such as the control interface, the moveable panel, or both, when the liner is positioned on the housing. In various embodiments, the liner covers an operational portion of the housing.

In various embodiments, the liner includes printed information. The printed information may relate to instructions for operating the medical device. The printed instructions may relate to a warning, safety information, instructions, or a combination of the same. In various embodiments, the housing is a control unit for use with a therapy wrap for applying to an anatomical body part. The liner may include printed information related to instructions for applying the therapy wrap to the body part. In various embodiments, the liner obscures or covers printed information on the housing.

In various embodiments, the liner comprises an opening corresponding to a handle on the housing. In various embodiments, the liner comprises an opening permitting access to the control interface. A perimeter of the opening may include printed instructions or indications for the control interface. The position of the opening may correspond to a position of a dial or setting on the control interface when the liner is positioned on the housing.

In various embodiments, the liner comprises markings at selected positions on the liner. In various embodiments, when the liner is positioned on the housing, the markings indicate information related to operation of the medical device.

In various embodiments, the liner is a laminate structure. In various embodiments, the liner is formed from a flexible material selected to lay flat and fold to conform to at least two adjacent sides of the housing. The liner may be plastic. The liner may be paper.

In various embodiments, the liner includes a booklet having fold-out panels for displaying information to a user.

In various embodiments, the liner includes printed information related to instructions for operating the control unit using the control interface. The printed information may be printed on the liner in a position corresponding to the moveable panel. The liner may include printed information related to instructions for operating the chamber. In various embodiments, the liner includes printed information in different languages. In various embodiments, the liner includes printed information in one language, for example English, and a translation of the information in another language. In various embodiments, the liner includes a panel configured to move with the moveable panel.

In various embodiments, the housing is a control unit for a thermal therapy system. The thermal therapy system may include a therapy wrap. The therapy wrap may include a fluid bladder and a compressive bladder for applying a compressive force to the fluid bladder against an anatomical body part. The fluid bladder and compressive bladder may be fitted within a sleeve. In various embodiments, the compressive bladder is an air bladder.

In various embodiments, the barrier extends around an entire circumference of the housing and/or control unit. In various embodiments, the barrier includes a panel for covering a surface of the housing having a control interface. The panel may be sized and dimensioned to cover all of the side surface of the housing. The panel may be sized and dimensioned to cover only a portion of the side.

Various aspects of the invention are directed to a method of operating a patient cooling and/or heating system, or a thermal therapy system. The method includes applying a therapy device to a patient, removing a portion of a barrier from a control unit for use with the therapy device, and acting based on the printed information.

In various embodiments, the removed portion or another portion adjacent the removed portion includes printed information related to operation of the control unit.

In various embodiments, the therapy device is a therapy wrap having a fluid bladder, the method further comprising circulating a heated or cooled fluid through the fluid bladder. The acting may include controlling circulation of the fluid based on the printed information.

In various embodiments, the removing comprises completely removing the portion. The removed portion may be a hinged panel. The removing may include opening the hinged panel.

Various aspects of the invention are directed to a method of administering a temperature-controlled treatment to an anatomical body part requiring treatment including applying a therapy device to a patient, providing a control unit having a control interface and a liner for use with the wrap, connecting the therapy device to the control unit using the control interface, and administering therapy to the patient using the control unit. In various embodiments, the control interface includes at least one of a control, a fluidic port, and an electrical port. In various embodiments, the liner covers a surface of the control unit having the control interface such that the control unit is rendered at least partially inoperative. In various embodiments, the connecting comprises moving or removing at least a portion of the liner to render the control unit operative. In various embodiments, the connecting comprises moving or removing at least a portion of the liner to access the control interface.

In various embodiments, the therapy device is a therapy wrap and the administering comprises flowing a heat exchanging fluid from a reservoir in the control unit to the therapy wrap through the control interface.

In various embodiments, the connecting comprises connecting an electrical connector from the therapy device to the control interface, and wherein the administering comprises controlling an electrical component of the therapy device using the control unit.

In various embodiments, the control interface includes an access port connected to an interior chamber of the control unit. The connecting may include connecting the therapy device to the access port. In various embodiments, the control interface includes an access panel providing access to an interior chamber of the control unit. The connecting may include moving the liner to access the access panel and opening the access panel.

In various embodiments, the connecting comprises removing a cut-out panel from the liner covering the control interface.

Various aspects of the invention are directed to a therapy device including a housing having a control interface on a side of the housing and a product label for preventing access to the control interface. In various embodiments, the product label is sized to cover the control interface side and at least another side of the housing.

In various embodiments, the product label includes a cover panel attached to the control interface side and covering the control interface to prevent access to the control interface, and another panel connected to the cover panel along an edge and covering the another side. In various embodiments, the product label comprises a planar base member and an adhesive for attaching the base member to the housing. In various embodiments, the product label includes a removeable release backing for exposing the adhesive.

In various embodiments, the product label includes a cut-out panel positioned over the control interface. In various embodiments, the cut-out panel is attached to the remainder of the label by at least one perforated edge. In various embodiments, the cut-out panel is attached to the remainder of the label by a hinge line. In various embodiments, all the edges of the cut-out panel attaching the panel to the remainder of the label are perforated.

In various embodiments, the cover panel includes printed information. In various embodiments, the cover panel includes printed information on a top surface opposite the housing surface. In various embodiments, the cover panel includes printed information on a bottom surface adjacent the housing surface.

In various embodiments, the control interface comprises a member selected from an interior access port, a fluidic connector, an electrical connector, a control, a display, and a combination of the same. In various embodiments, the housing is a housing for a control unit for a thermal therapy device.

Various aspects of the invention are directed to a device comprising any of the features described in paragraphs [0007] to [00030]. Various aspects of the invention are directed to using the device. In various embodiments, the method comprises removing a barrier or label from the housing to enable a user to access the control interface and/or control unit. In various embodiments, the method comprises moving at least a portion of a barrier or label to expose the control interface and/or control unit. In various embodiments, the method comprises lifting a cut-out portion to expose the control interface and/or control unit. In various embodiments, the method comprises reading printed information on the label before exposing the control interface and/or control unit. In various embodiments, the method comprises operating the device based on the printed information.

The devices of the invention have other features and advantages which will be apparent from or are set forth in more detail in the accompanying drawings, which are incorporated in and form a part of this specification, and the following Detailed Description of the Invention, which together serve to explain the principles of the present invention.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which.

In the drawings:

FIG. 1 is a view of the components in an exemplary therapy system.

FIG. 2 is a perspective view of a control unit barrier intended to prevent access to the interior of a control unit. The barrier is shown attached to a control unit.

FIG. 3 is a perspective view of a control unit barrier intended to prevent access to the interior of the control unit, the control interface and the power connector. The barrier is shown attached to a control unit.

FIG. 4 is a view of an example of a control unit barrier indicating the location of information on the barrier.

FIG. 5 is a view of an example of a control unit barrier having an attachment strap.

FIG. 6 is a view of an example of a control unit barrier with attachment material used to attach it to a control unit.

FIG. 7 is a view of an example of a control unit barrier and a barrier attachment strap used to affix the barrier to the control unit.

FIG. 8 is a view of an example of a connector barrier in place on the end of a connector hose around the hose connector.

FIG. 9 is a view of an example of a connector barrier in place in the end of a connector hose covering the hose connectors.

FIG. 10 is a view of an example of a control unit barrier with a pouch and an access card sized to fit within the pouch.

FIG. 11 is a view of the control unit barrier and access card of FIG. 10 in place around a control unit.

### DETAILED DESCRIPTION OF THE INVENTION

Before the present invention is described, it is to be understood that this invention is not intended to be limited to particular embodiments or examples described. Further, when referring to the drawings, like numerals indicate like elements.

Various aspects of the invention are similar to the subject matter described in: U.S. patent application Ser. No. 09/127,256 (filed Jul. 31, 1998) entitled, “Compliant Heat Exchange Panel” issued on Apr. 3, 2007 as U.S. Pat. No. 7,198,093; U.S. patent application Ser. No. 09/798,261 (filed Mar. 1, 2001) entitled, “Shoulder Conformal Therapy Component of an Animate Body Heat Exchanger” published on Aug. 30, 2001 as U.S. Publication No. 2001-0018604A1; U.S. patent application Ser. No. 09/901,963 (filed Jul. 10, 2001) entitled, “Compliant Heat Exchange Splint and Control Unit” published on Nov. 8, 2001 as U.S. Publication No. 2001-0039439A1; U.S. patent application Ser. No. 09/771,123 (filed Jan. 26, 2001) entitled, “Wrist/Hand Conformal Therapy Component of an Animate Body Heat Exchanger” published on Oct. 25, 2001 as U.S. Publication No. 2001-0034546A1; U.S. patent application Ser. No. 09/771,124 (filed Jan. 26, 2001) entitled, “Foot/Ankle Conformal Therapy Component of an Animate Body Heat Exchanger” published on Feb. 14, 2002 as U.S. Publication No. 2002-0019657A1; U.S. patent application Ser. No. 09/771,125 (filed Jan. 26, 2001) entitled, “Conformal Therapy Component of an Animate Body Heat Exchanger having Adjustable Length Tongue” published on Oct. 25, 2001 as U.S. Publication No. 2001-0034545A1; U.S. patent application Ser. No. 10/784,489 (filed Feb. 23, 2004) entitled, “Therapy Component of an Animate Body Heat Exchanger” published on Aug. 26, 2004 as U.S. Publication No. 2004-0167594A1 which is a continuation of U.S. patent application Ser. No. 09/765,082 (filed Jan. 16, 2001) entitled, “Therapy Component of an Animate Body Heat Exchanger and Method of Manufacturing such a Component” issued on Feb. 24, 2004 as U.S. Pat. No. 6,695,872 which is a continuation-in-part of U.S. patent application Ser. No. 09/493,746 (filed Jan. 28, 2000) entitled, “Cap And Vest Garment Components Of An Animate Body Heat Exchanger” issued on Jan. 30, 2001 as U.S. Pat. No. 6,178,562; U.S. patent application Ser. No. 10/122,469 (filed Apr. 12, 2002) entitled, “Make-Break Connector For Heat Exchanger” issued on Mar. 29, 2005 as U.S. Pat. No. 6,871,878; U.S. patent application Ser. No. 10/637,719 (filed Aug. 8, 2003) entitled, “Apparel Including a Heat Exchanger” issued on Sep. 19, 2006 as U.S. Pat. No. 7,107,629; U.S. patent application Ser. No. 12/208,240 (filed Sep. 10, 2008) entitled, “Modular Apparatus for Therapy of an Animate Body” published on Jan. 1, 2009 as U.S. Publication No. 2009-0005841A1 which is a divisional of U.S. patent application Ser. No. 10/848,097 (filed May 17, 2004) entitled, “Modular Apparatus for Therapy of an Animate Body” issued on Mar. 1, 2011 as U.S. Pat. No. 7,896,910; U.S. patent application Ser. No. 11/707,419 (filed Feb. 13, 2007) entitled, “Flexible Joint Wrap” issued on Nov. 23, 2010 as U.S. Pat. No. 7,837,638; U.S. patent application Ser. No. 11/854,352 (filed Sep. 12, 2007) entitled, “Make-Break Connector Assembly with Opposing Latches” issued on Jun. 8, 2010 as U.S. Pat. No. 7,731,244, the entire contents of which patents and publications are incorporated herein for all purposes by reference.

The above systems and components generally provide active heating, cooling, and/or compression for humans and other animal bodies. They are used, for example, in physical therapy, pre-game conditioning, minor injury care, post-operative care, and emergency medical care, among other applications. Thermal therapy systems exist in a number of different forms. In general, there is a control unit, a connector hose, a wrap comprising a heat transfer device and cover, and a power source (i.e., battery or externally-powered electric source). The therapy wrap comprising the cover and heat exchanger is applied to the portion of the mammal's body to receive therapy. The control unit modulates a heat transfer medium in the wrap to achieve the desired therapeutic result. One such system is disclosed, for example, in U.S. Pat. No. 6,178,562, the disclosure of which is herein incorporated for all purposes by reference.

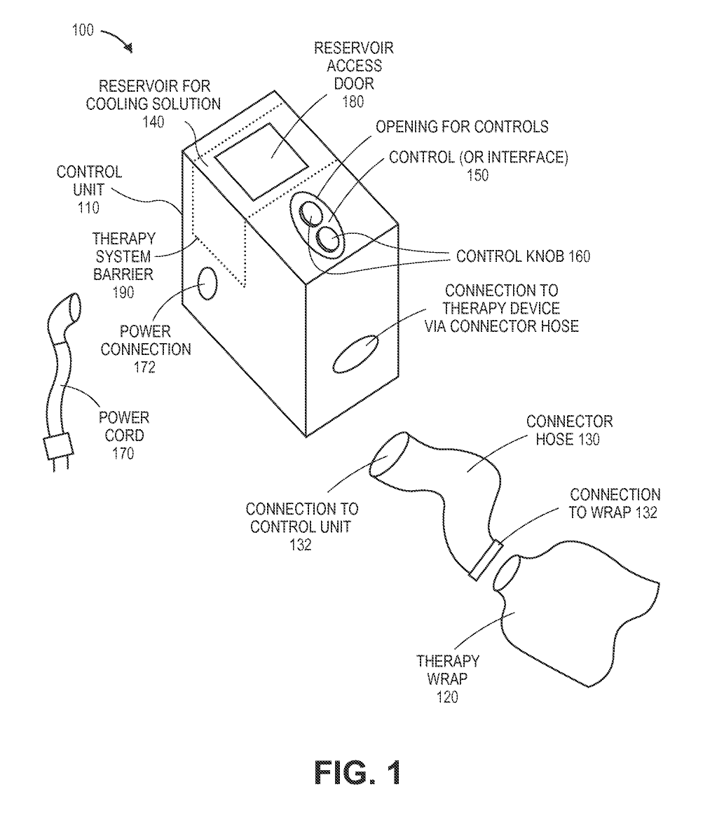

Turning to the drawings, FIG. 1 illustrates a representative number and type of components used in a conventional cold therapy system 100. The cold therapy system 100 includes a control unit 110 that is connected to a therapy wrap 120 using a connector hose 130 and connectors 132. Not shown but within the control unit 110 is a pump in communication with the reservoir 140 and the wrap 120. Under control of a control system within the control unit 110, the pump takes water from the reservoir 140 and circulates it through the wrap 120 after which it returns to the reservoir 140. Operation of the system 100 is provided using a suitable control interface. In the illustrated system 100 embodiment, a pair of control knobs 160 is provided. It is to be appreciated that a number of different control interfaces 150 could be used including, but limited to, recessed knobs that pop up for use, touch screens, or push buttons.

The connector hose 130 may have connectors 132 to mate with both the control unit 110 and the wrap 120 or the connector hose 130 may be permanently connected to the control unit 110 and/or the wrap 120. A power cord 170 is provided to connect the control unit 110 to a suitable power source through a power connection 172. In the illustrated system 100, a reservoir access door 180 is provided to allow water and ice to be placed into the reservoir 140. Some systems may use a different source of cooling for a cold therapy system, such as a thermo-electric cooler or a heating source suited for use in heating therapy systems.

FIG. 1 illustrates an exemplary therapy system 100 including a barrier or liner, generally designated 190. In various respects, “barrier” and “liner” are used interchangeably. In various respects, “barrier” and “product label” are used interchangeably.

Aspects of embodiments of the present invention relate to a therapy system barrier 190 and the use of such barriers. A therapy system barrier 190 is a device or structure that hinders proper operation of a related therapy system until the barrier 190 is removed. A therapy system barrier 190 interacts with one or more therapy system components to hinder proper use or operation of the involved therapy system component. The barrier 190 is modified depending upon the component or components involved.

The exemplary control unit 110 includes a control interface, generally designated 150. As used herein, in various respects “control interface” is to be understood broadly as including various components for operation of the control unit 110. The control interface may include, but is not limited to, control knobs 160, a display, a fluidic connection, and electrical connection, an access panel, a memory device interface, and more.

In related and additional aspects, a barrier includes information and/or instructions as discussed further below. Information and/or instructions are provided to assist a user in the safe, proper and/or intended therapy system use. The information or instructions may be provided in a way so as to assist a user when using a therapy system for the first time. For example, the instructions may include “Getting Started” or “Before First Use” instructions. The barrier may include other printed information such as warnings, promotions, product graphics, and more. Additionally or alternatively, the information or instructions may be provided in a way to assist a user in subsequent, ongoing or periodic therapy system use. Moreover, information or instructions are placed on or presented by a barrier in a conspicuous manner. Conspicuous placement of information and/or instructions increases the likelihood that a user will notice, interact with, comply with or modify behavior in accordance with the information and/or instructions.

A therapy system barrier may take many forms. In various embodiments, the barrier is a flexible material for wrapping around or conforming to the control unit surface. The barrier may be formed of conventional materials and constructions as would be understood by one of skill from the description herein. In various embodiments, the barrier has a laminate construction. The barrier 190 may include a substrate and an adhesive backing. The barrier 190 may include a top layer over the substrate. Suitable materials for the barrier 190 include, but are not limited to, plastic and paper (e.g. cardboard). In various embodiments, the barrier 190 is configured similar to a sticker or product graphic.

Broad categories for grouping barriers include, for example, electrical barriers and mechanical barriers.

An electrical barrier would be one that interferes with the power supply to the system, the control panel or interface or to those components under the control of a control system or an operating panel, for example. Electrical barriers also include those techniques that inhibit or interfere with the inner working of the system. Examples of electrical barriers include software or firmware access control programs or applications that would require a user to enter or provide a pass code, an authorization code or a use code that interacts with software, firmware or other control system components. The user may provide the pass code using any of a number of different techniques. One example would be the keypad entry of a pass code or key phrase. Another example would be the use of a card reader authorization system. Another example would be the use of a card access control system. An access or authorization system may include any conventional access or control method such as, for example, barcode, magnetic stripe, biometric, proximity, contact smart card, contactless smart card or PIN. Additionally or alternatively, a non-conducting card may be inserted between the power source and the controller or another component of the therapy system. The presence of the non-conducting card prevents power from being supplied to a specific component or, alternatively, to the therapy system in general. Power is not provided to the system or component until the non-conductive card is removed.

Another broad category of barrier is a mechanical barrier. A mechanical barrier is a barrier that physically attaches to or prevents component use. In some cases, a physical barrier is removed to permit component operation. In other words, a user is required to remove or move the barrier to gain access to necessary controls and components for operation of the device. In other configurations, the physical barrier remains attached to the component but is displaced to permit component use. The barrier may include a moveable panel for gaining access to a portion of the control unit. For example, the barrier may include a flap, a panel that slides or translates along a track, or a rotating member like a viewing window.

In general, one or more barriers may be utilized with the involved component or components in any of a number of suitable ways depending on the barrier and the component. Examples of barrier placement and/or interaction include but are not limited the following examples.

A barrier may be placed on, positioned about, or joined to the control unit to hinder use of one or more control unit functions.

A barrier may be positioned with a component in a way whereby the entire barrier is removed for one time use (i.e., barrier is removed but not replaced) or, alternatively, where the barrier removed but is available for repeated use (i.e., barrier is removable but may be re-attached to component(s)).

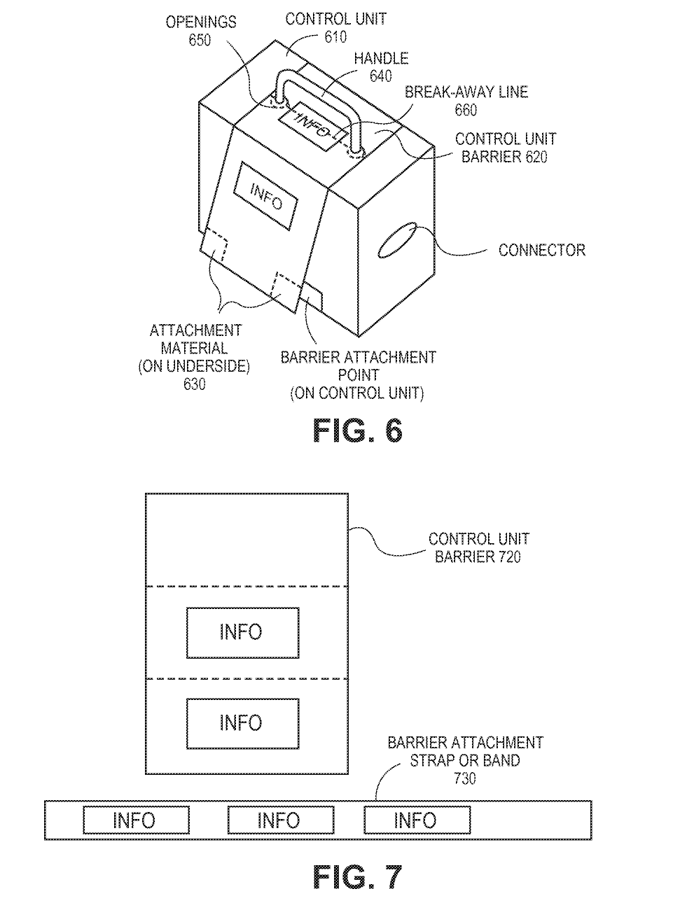

In another alternative, the barrier remains attached to one or more components but component operation is permitted only after modification of the barrier. Modification may be reversible or one time. Reversible modifications include, for example, the use of adhesives that may be removed and then reapplied. Examples include joining the barrier with a controlled force adhesive, multiple use adhesive, zip lock, tie, hook and loop or other re-useable closure or attachment technique. Alternatively, the barrier may remain attached to the component but be folded back to permit access to or use of the component or system. In various embodiments, the barrier includes a one panel for covering a side of the control unit having a control interface and another panel. The one panel and the another panel are connected by a fold line. The panels may be sized to cover all or a portion of a side surface of the control unit. The fold line may be positioned to align with an edge of the control unit such that the panels fold over two sides of the control unit (e.g., as shown in FIGS. 6 and 7).

One time barrier use is barrier design where a portion of the barrier is cut, torn, removed (completely or partially) or otherwise damaged so as to then permit operation or use of the involved component(s) via the torn, damaged or removed portion of the barrier.

In another alternative, the barrier is attached to the component or to the system by folding it over the component or by attaching to the component with a strap, band or other attachment type described above. This form of attachment may be removable without damage to the barrier and the barrier may be replaced after component or system use.

While the barrier may be designed to inhibit improper use and foster proper use after taking recommended steps, another aspect of a barrier is to provide information about the proper use of the therapy system generally or for the operation of the therapy system for a particular user (i.e., operation to provide a specific therapy or treatment plan for an individual user as in a prescribed course of therapy, including for example physical therapy or rehabilitation program). In an exemplary embodiment, the barrier includes one or more openings positioned and sized for receiving components of the control unit (e.g., control knobs and a reservoir lid as shown in FIG. 1). The printed information may be positioned adjacent the openings such that it will be conspicuous to a user during use. Information may be provided on a top or bottom of the barrier. For example, information may be printed on the bottom of a cut-out or flap such that the information is revealed when a user modifies the barrier before use. In the case of a laminate barrier, information may be provided on any of the layers.

Non-limiting examples of information associated with the barrier include:

Information about how to properly set up the therapy system—the order of attaching components or performing steps, for example. The information may also include operational instructions for proper and intended use of the system, such as for a specific patient therapy.

Information may also include safety information.

Information may also include one or more operational codes for the proper use of the system or one or more components. One such code is a code that is not seen until a barrier is removed or repositioned as a way to ensure that the user's attention is directed to the information on the barrier.

Information may also include words or images presented on a surface seen while the barrier is hindering operation of a component or system. Information may be pre-printed on a barrier or space may be provided for adding information to the barrier. Examples of pre-printed information include, but are not limited to: a customer satisfaction form, a warranty card, warning and/or caution statements related to therapy system use, marketing promotions, an operator manual or instructions for set up and/or operation of the system, a component, more than one component or combinations thereof. Information may be added to a barrier writing or printing onto a designated area of the barrier. Additionally or alternatively, a pouch, envelope or other container may be provided on the barrier to hold additional information. The pouch or envelope may contain any of a wide variety of information as described herein. Non-limiting examples include a comment card, a user authorization card, user authorization code or card, user specific information such as contact information or therapy settings or prescribed treatment program to be used with the therapy system. In various embodiments, the outer surface of the pouch is transparent so the contents are viewable by a user. In various embodiments, the pouch is positioned over a operative component of the control unit.

Information may also include a patient's name, contact information, physician and/or physical therapist information, a patient's prescription or a summary of a patient treatment plan or prescription. The information may be written directly onto or printed directly onto a barrier. Alternatively, the information may be written, printed or otherwise contained in a human or machine tangible medium for use by a component, components, the system or combinations thereof. The information may be placed on an adhesive label and attached to a component. The information may be placed in an envelope on the barrier for that purpose. In another alternative, the envelope is clear or transparent so that the information may be within the envelope but still visible. In various embodiments, the barrier includes a fold-open pamphlet with information related to operation of the device and/or product information. For example, the barrier may a base panel and one or more fold-out panels connected to the base panel along a fold line. The fold-out panels may open like a booklet or in an accordion style. The pamphlet may be resealable.

Alternatively, the information is contained on a user access device coded with component, system or user specific information. Examples include user identifying information coded into any of the electrical barriers described above. This information may include prescription information, system instructions based on prescription information, payment authorization information, and period of use limitations, date restrictions on system or component operation or number of permitted system therapy cycles. One form of specific electronic barrier is a user information coded access card that is coded with user specific information, component specific information (such as configuration) or operating settings, or payment information, or any of the other information listed herein. In addition, one or more components of the system (such as the control unit) may be modified to include an appropriate reader to receive and/or exchange information with a user coded electronic barrier.

In one specific example, the information is added to the barrier in an appropriate form (i.e., written, printed or coded into electronic form or onto a user access device) in a physician office when the therapy is prescribed. Adding information in this context refers to the prescribing physician or his staff or designee providing patient specific information in a form appropriate for use with the barrier. Examples of how this step or act may be accomplished include, but are not limited to: writing information on a barrier, completing a patient data card configured for use with the barrier and affixing it to the barrier, completing a patient data card and placing it into a pouch or envelope provided on the barrier, attaching patient data to or indicating on a strap that holds the barrier in place over, on or with a component. Additionally or alternatively, the physician, staff or designee may load, program, write, activate or authorize an access system, card or device for a specific user to use a specific device within a physician prescribed or determined range.

In one specific example, the physician, staff or designee activates a user control card with user specific information and treatment prescription, places the activated card into an appropriate pouch on the barrier and then attaches the barrier to the therapy system. In one alternative, the physician, staff or in the physician's office, the card is authorized and attached to a barrier for later attachment to a therapy system. In another alternative, the physician actives a user access card as discussed above and provides that card to the patient. The patient then takes the access card to a service provider who does one or more of the following steps: (a) confirms the validity of the user information including identification, payment information, period of authorized usage, number of authorized uses or other information for that patient or system; (b) ensures that a prescription is included or indicated; (c) ensures physician information is included; (c) loads therapy system specific information based on information from the physician; and/or (d) provides amplifying information to patient based on prescription information.

These and other aspects of the various embodiments of the invention will be appreciated in the following examples.

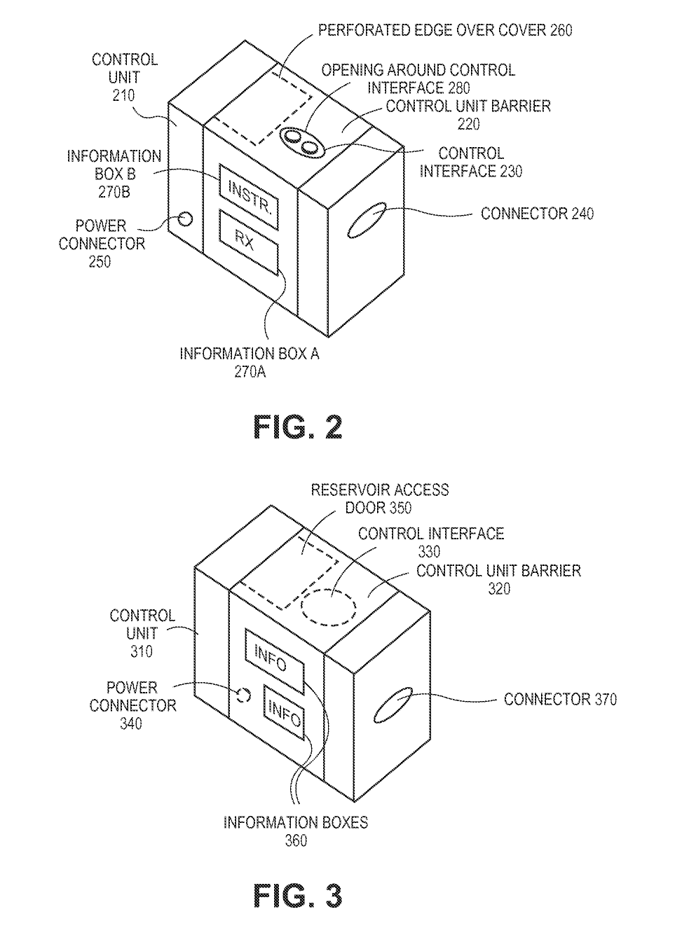

FIG. 2 is a perspective view of a control unit barrier 220 intended to prevent access to the interior of a control unit 210. The barrier 220 is shown attached to a control unit 210 similar to the control unit 110 of FIG. 1. The barrier 220 includes an opening around control interface 280 that permits access to the control interface 230. The barrier 220 is around at least two sides of the control unit 210 and does not interfere with either the connector 240 or the power connector 250. The barrier 220 completely covers the access to the reservoir within the control unit 210. An exemplary perforated line 260 is provided that aids in removal of a portion or all of the liner to permit access to the reservoir hatch or cover. Two information boxes 270 (A and B) are shown on the barrier 220. The information boxes 270 may include any of the various forms of information described herein. In this example, information box 270b contains information about how to operate the system or system instructions. Information box 270a contains information about or a copy of the patient's prescription for using the device in a particular therapy regime.

FIG. 3 is a perspective view of a control unit barrier 320 intended to prevent access to the interior of the control unit 310, the control interface 330 and the power connector 340. The barrier 320 is shown attached to a control unit 310. In contrast to the control unit barrier 220 in FIG. 2, the illustrated control unit barrier 320 inhibits use of the control unit 310 functions of power connection 340, control interface 330 operation and/or reservoir access 350. The location of each of these functions is shown in phantom to indicate their location under the barrier 320. As before, the exemplary control unit barrier 320 provides one or more information boxes 360 containing information as described herein. The only control unit 310 functionality available while the barrier 320 is in place is the connection port 370 to the therapy wrap.

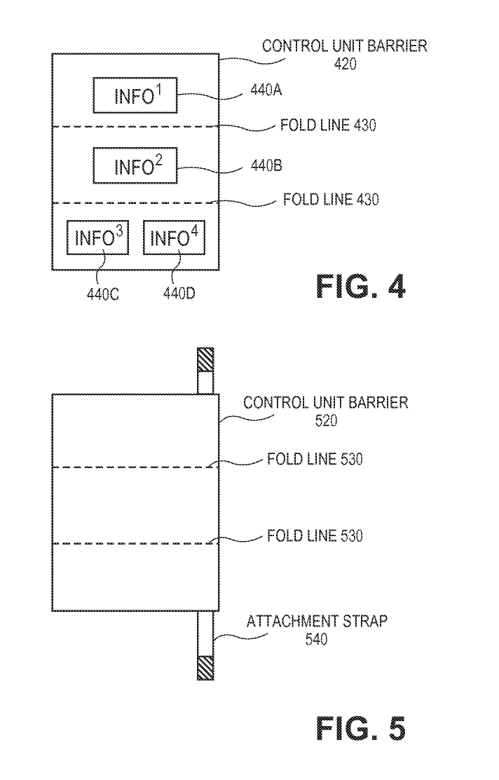

FIG. 4 is a view of an example of a control unit barrier 420 indicating the location of information on the barrier 420. The view in FIG. 4 provides a better view of a control unit barrier 420 that is folded to enclose the control unit. By folding a barrier 420 along one or more fold lines 430, the barrier 420 can conform to all or a portion of the exterior surface of a component. In the illustrated example, the barrier 420 is intended to function with a generally rectangular control unit. The example of a regularly shaped component is merely for purposes of example. A barrier may take any of a variety of shapes to conform to the outside surface of a component. A component may have a regular shape such as the illustrated rectangular box or a cylinder, for example. Components may also have an irregular shape or surface. For irregularly shaped components, the barrier is modified accordingly. Four information boxes 440 are shown on the exemplary control unit barrier 420. More or fewer information boxes 440 may be provided and information may also be provided on one or both sides of the barrier 420. In some embodiments, the information printed on the side of the barrier 420 adjacent to the component is not visible to the user until the barrier 420 is removed completely or partially from the component or an indicated portion of the barrier 420 is removed (i.e., via tear away, cut away, pull to release or open lines or through the use of perforations as shown above).

FIG. 5 is a view of an example of a control unit barrier 520 having an attachment strap 540. In this embodiment the barrier 520 relies not only on folding along one or more fold lines 530 to conform its shape to the exterior of a component but also include the use of an integrally formed or attached strap 540. The strap 540 includes suitable fasteners on the ends such as adhesives, hook and loop, buckles or other joining devices.

FIG. 6 is a view of an example of a control unit barrier 620 with attachment material 630 used to attach it to a control unit 610. In contrast to the strap 540 of FIG. 5, adhesive could be applied to the outside of the component and the barrier 620 attached directly to the component. In the illustrative example, the barrier 620 is attached directly to the control unit 610 using an adhesive on the underside of the barrier 620 and also on the control unit 610. Alternatively, the adhesive could be on one of the barrier 620 or the component or the barrier 620 may be joined to the component using any of the two part fasteners described above or known to those of ordinary skill.

The exemplary control unit 610 includes a housing having a handle 640. The exemplary barrier 620 having an opening corresponding to the handle 640. The exemplary barrier 620 includes two openings 650 for receiving the handle 640 and a break-away line 660 for allowing a user to tear away the barrier 620 from the housing and handle 640. The exemplary break-away line 660 is a perforated cut line in the barrier 620 material. In various embodiments, the barrier 620 is configured so the user removes the entire barrier 620 from the housing to gain access to and operate the control unit 610. In various embodiments, the barrier 620 is configured so a user removes only a portion of the barrier 620. In one exemplary embodiment, the barrier 620 includes a cut-out over a control interface of the control unit 610, the cut-out being configured for easy removal by a user.

In an additional alternative embodiment, a barrier is attached to a component in a manner that permits the use of a tamper evident seal. In this case, if a barrier is improperly removed or replaced, then there is detectable indicium of such removal or replacement. In various embodiments, the barrier encloses the control unit such that its removal serves as a visual indication to a user that the control unit has been used already (shown, e.g. in FIGS. 6 and 7). A tamper evident barrier attachment technique may be used to detect if a component, such as a control unit or a wrap, was prescribed for one patient for one specific therapy but now is attempted to be used for a different patient or for a therapy that is different from the originally prescribed therapy.

FIG. 7 is a view of an example of a control unit barrier 720 and a barrier attachment strap 730 used to affix the barrier 720 to the control unit. In this illustrative embodiment, a separate strap 730 or band is used to retain a barrier 720 on a component. The band or strap 730 may be particularly useful for different shaped components. In one alternative, the band 730 is elastic and is used much like a rubber band to hold the barrier 720 in place. In another alternative, the elastic band 730 contains information used in conjunction with the barrier 720 or with one or more components. In another alterative the strap 730 contains information used to operate the system and the information is provided on the strap 730 so that the only way to see the information is to remove the strap 730 from the component. The strap 730 may also be used as a tamper evident indicator on one or more components.

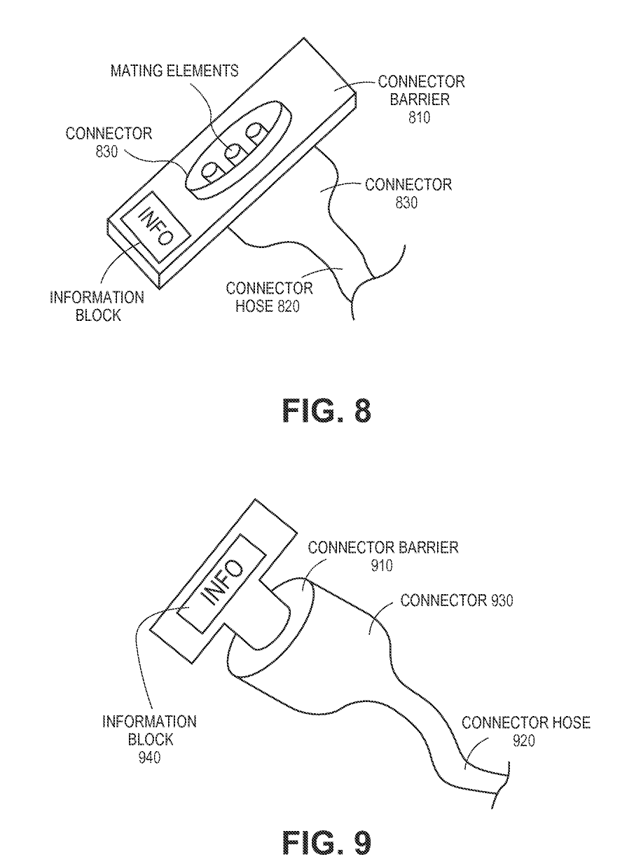

FIG. 8 is a view of an example of a connector barrier 810 in place on the end of a connector hose 820 around the hose connector 830. In addition to the control unit, any component may be provided with a suitable barrier. In the illustrative embodiment of FIG. 8, the barrier 810 is a connector barrier 810 that—so long as the connector barrier 810 remains in place as shown—inhibits the connection between the supply line and the control unit or the wrap. A similar connector barrier 810 may be used with the power connection as well.

FIG. 9 is a view of an example of a connector barrier 910 in place in the end of a connector hose 920 covering the hose connectors. As with FIG. 8, the illustrative embodiment of FIG. 9 is shown in use with a connector 930. In the illustrative embodiment, the connector barrier 910 is inserted into a connector 930. So long as the connector barrier 910 remains in place, the connector 930 cannot be attached to the control unit or the wrap. As with the connector barrier 810 in FIG. 8, the connector barrier 910 may also contain information about the component, the system, the therapy or any other farm of information described herein.

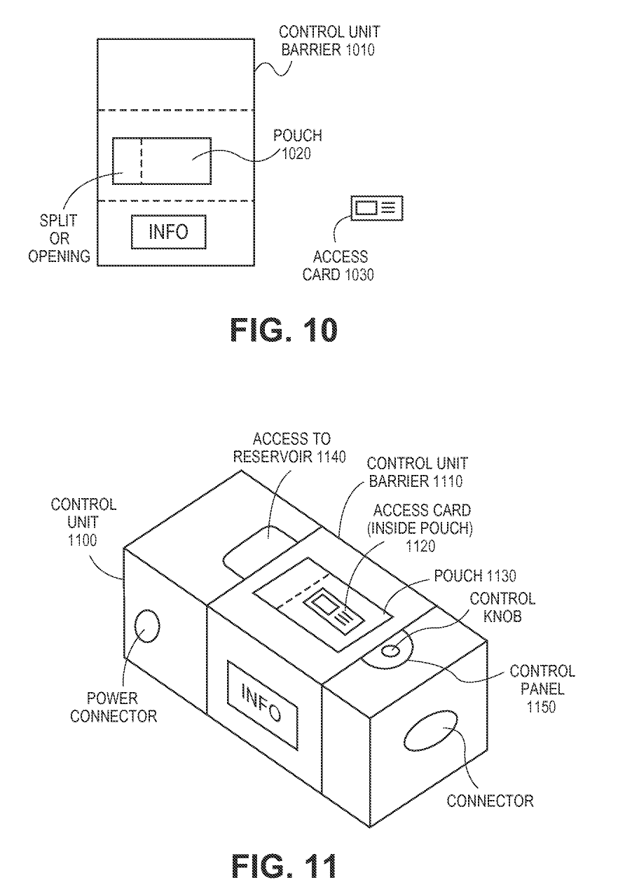

FIG. 10 is a view of an example of a control unit barrier 1010 with a pouch 1020 and an access card 1030 sized to fit within the pouch 1020. The illustrative barrier 1010 contains a pouch 1020 that may be used to convey information as described herein. An access card 1030 is also illustrated that may be coded with information as described above and then placed into the pouch for a barrier going to a specific patient.

FIG. 11 is a view of the control unit barrier 1110 and access card 1120 of FIG. 10 in place around a control unit 1100. In this illustrative embodiment, the access card 1120 has been coded with patient specific information and the access card 1120 placed into the pouch 1130 on the barrier 1110. The barrier 1110 in turn has been fixed to the control unit 1100 using any of the above described techniques. In this embodiment, the barrier 1110 is inhibiting the access to the reservoir 1140 as well as access to the control interface 1150. Moreover, as described above, the access card 1120 may also inhibit the use of the control unit 1100 or other components according to the information coded into the access card 1120. It is to be appreciated that the components or systems described herein intended for use with any of the various access card types and associated systems, contain the appropriate readers, scanners, software, firmware or other electronic components so that the information—however stored in the access card—is made available to component or system as needed.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed is:

1. A medical device, comprising:
a housing;
an access to an interior chamber of the housing;
a control interface on a surface of the housing; and
a liner sized to cover at least a portion of the surface of the housing, wherein when the liner is positioned to cover the surface of the housing, the access is rendered at least partially inoperative.

2. The device of claim 1, wherein the liner is sized to cover the portion of the surface and at least one other face of the housing.

3. The device of claim 1, wherein the liner includes printed information related to warnings, safety information, instructions, or a combination of the same for operating the medical device.

4. The device of claim 3, wherein the printed information comprises information in different languages.

5. The device of claim 1, the housing being a control unit for use with a therapy wrap, wherein the liner includes printed information related to instructions for applying the therapy wrap to a body part.

6. The device of claim 1, wherein the liner comprises an opening corresponding to a handle on the housing.

7. The device of claim 1, wherein the liner comprises an opening permitting access to the control interface.

8. The device of claim 7, wherein a perimeter of the opening includes printed instructions or indications for the control interface.

9. The device of claim 7, wherein the position of the opening corresponds to a position of a dial or setting on the control interface when the liner is positioned on the housing.

10. The device of claim 1, wherein the liner comprises markings at selected positions on the liner, and wherein when the liner is positioned on the housing, the markings indicate information related to operation of the medical device.

11. The device of claim 1, wherein the liner is formed from a flexible material selected to lay flat and fold to conform to at least two adjacent sides of the housing.

12. The device of claim 11, wherein the liner is plastic.

13. The device of claim 11, wherein the liner is paper.

14. The device of claim 1, wherein the liner includes a booklet having fold-out panels for displaying information to a user.

15. The device of claim 1, wherein when the liner is positioned to cover the surface, the access is rendered completely inoperative.

16. A control unit for a patient therapy system, comprising:
a housing containing the control unit;
a chamber within the housing;
an interface on the housing used to interact with the control unit;
a moveable panel on the housing in communication with the chamber; and
a liner sized to conform to and at least partially cover a surface of the housing, wherein the liner renders inoperative the control interface, the moveable panel, or both when the liner is positioned on the surface.

17. The control unit of claim 16, wherein the liner sized to conform to and at least partially cover at least two adjacent surfaces of the housing.

18. The control unit of claim 16, wherein the liner renders completely inoperative the control interface, the moveable panel, or both when the liner is positioned on the surface.

19. The control unit of claim 16, wherein the liner includes printed information related to warnings, safety information, instructions, or a combination of the same for operating the control unit using the control interface.

20. The control unit of claim 17, wherein the printed information is printed on the liner in a position corresponding to the moveable panel.

21. The control unit of claim 16, wherein the liner includes printed information related to instructions for operating the chamber.

22. The control unit of claim 16, wherein the liner includes a panel configured to move with the moveable panel.

23. A method of operating a patient cooling and/or heating system, comprising:
applying a therapy device to a patient;
removing a portion of a barrier from a control unit for use with the therapy device, the removed portion or another portion adjacent the removed portion including printed information related to operation of the control unit; and
acting based on the printed information.

24. The method of claim 23, wherein the therapy device is a therapy wrap having a fluid bladder for circulating a heat exchange medium.

25. The method of claim 24, wherein the acting comprises controlling circulation of the heat exchange medium based on the printed information.

26. The method of claim 23, wherein the removing comprises completely removing the portion.

27. The method of claim 23, wherein the removed portion is a hinged panel, and the removing comprises opening the hinged panel.

28. A method of administering a treatment to an anatomical body part requiring treatment, comprising:
applying a therapy device to a patient;
providing a control unit having a control interface and a liner for use with the wrap, the control interface including at least one of a control, a fluidic port, and an electrical port, wherein the liner covers at least a portion of a surface of the control unit having the control interface such that the control unit is rendered at least partially inoperative;
connecting the therapy device to the control unit using the control interface, wherein the connecting comprises moving or removing at least a portion of the liner to render the control unit operative; and
administering therapy to the patient using the control unit.

29. The method of claim 28, wherein the therapy device is a therapy wrap and the administering comprises flowing a heat exchanging fluid from a reservoir in the control unit to the therapy wrap through the control interface.

30. The method of claim 28, wherein the connecting comprises connecting an electrical connector from the therapy device to the control interface, and wherein the administering comprises controlling an electrical component of the therapy device using the control unit.

31. The method of claim 28, the control interface comprising an access port connected to an interior chamber of the control unit, wherein the connecting comprises connecting the therapy device to the access port.

32. The method of claim 28, wherein the connecting comprises removing a cut-out panel from the liner covering the control interface.
